(19) United States
(12) Patent Application Publication
(10) Pub. No.: US 2010/0319709 A1
(43) Pub. Date: Dec. 23, 2010

(54) DEVICE FOR TREATMENT OF SNORE AND APNEA AND FOR USE AS MOUTH PROTECTOR

(76) Inventor: Kenya Carla Felicissimo Gonçalves, Itaigara (BR)

Correspondence Address: STITES & HARBISON PLLC, 1199 NORTH FAIRFAX STREET, SUITE 900, ALEXANDRIA, VA, 22314, US

(21) Appl. No.: 12/760,903

(22) Filed: Apr. 15, 2010

(30) Foreign Application Priority Data
Apr. 15, 2009 (BR) PI-0901237-0

(51) Int. Cl. A61F 5/56

(52) U.S. Cl. 128/848

(57) ABSTRACT

A device is provided to be used to treat sleep deprivation disorders, such as apnea, snore, and bruxism, among others, for patients with mouth respirators and jawbone disorders; and also be used as mouth protectors. The device is manufactured as a two-part piece or as a single piece; and is fit to position by the patient or trained professional, therefore allowing progressive anterior-posterior fits and providing for side movements.

### FIELD OF THE INVENTION

The present invention is in the field of devices for treatment of sleep deprivation disorders.

### BACKGROUND OF THE INVENTION

Intra-Oral (AIO) devices available today for treating sleep deprivation disorders have some drawbacks such as high cost, difficulty to adapt by the patient, the material used.

It is believed that the first description of the Sleep Obstructive Apnea Syndrome (SAOS) was published on “Pickwick Papers” and that the first medical description was published in 1956 by Burwell et al. as reported by Kryger (1985) and Tjoostad (1995). The advancement of sleep physiology and more accurate record techniques, and the growing acknowledgement of sleep pathologies today allow the Medicine of Sleep to be established as a new medical specialty. Hall & Bradley reported that in 1995, SAOS was related to heart conditions and pointed that the joint treatment of heart problems and SAOS can lead to improved cardiovascular function.

Parker, in 1995, reported that SAOS patients usually have a narrowing of their VAS (upper airways), which is usually located in the soft palate terminal region and base of the tongue. It is believed that mouth obstruction takes place at the base of the tongue. The narrowing of the pharyngeal airway is caused by two main factors: a reduction of the genioglossal muscle and of muscles close to VAS, as well as the volume of tissues close to VAS (tonsils, uvula, soft palate, etc.). VAS muscles and genioglossal muscle are responsible for keeping the caliper of our airways. This standard is met by balancing these muscles with a negative intra pharyngeal pressure that occurs during inhaling. This reduction of pharyngeal muscle and genioglossal muscle tonicity causes VAS to become narrower, which causes increased airflow speed, therefore generating a vibration of the soft palate and pharynx tissues and producing snore. VAS narrowing increases the intra pharyngeal pressure, which, when associated to a reduction to the genioglossal muscle tonicity, creates a condition where the tongue is sucked against the front wall of the airways, causing and obstruction. By definition, if such obstruction lasts longer than 10 seconds, apnea takes place. The base of the tong is the site where, most frequently, obstruction takes place, according to Parker. Events take place in the following sequence: When obstructive apnea takes place, oxygen level in the blood is reduced and, conversely, the level of carbon dioxide in the brain increases. The respiratory center in our brain detects these changes and responds by increasing the tonicity of the genioglossal and pharyngeal muscles, letting air flow in, however, under pressure, it is similar choking, awakening the individual from his deep sleep. This awakening fragments our sleep, leading to hypersonolence during the day, a usual symptom in patients who suffer from SAOS.

How the disease is prevalent: George reports in his paper published in 1993 that families who don't have at least one snoring member are most certainly a minority. Authors writing on this subject estimate that approximately 60% of men and 40% of women snore (US). In his paper published in 1995, Partinen reports that Obstructive Sleep Apnea Syndrome (SAOS) is the most commonly occurring sleep disorder. He claims that according to epidemiological studies, SAOS prevalence is approximately 2% for women and 4% for adult men.

Approximately 50% of all adult men snore and about 10%. In 1988, Berry-Borowiecki et al. published a study which surveyed anatomic cranial differences among individuals who either did or did not suffer from SAOS. They studied 30 patients with SAOS and 12 patients without SAOS (control group) and used a cephalometric analysis. Statistical findings show that patients with Obstructive Sleep Apnea Syndrome were different from patients in the control group with regard to five main elements: 1) Tongue and soft palate are significantly larger; 2) The hyoid bone is inferiorly dislodged; 3) the jawbone is normal in its size and position, but the face is elongated due to a dislodgement of the lower jawbone body; 4) Maxilla is retropositioned and the soft palate is elongated and 5) the nasopharynx is normal but the aropharynx and hypopharynx airway is reduced to an area of 25% on average, a factor that could worsen SAOS symptoms.

In 1990, Lowe et al. published a study showing results from the use of a jawbone repositioning mouth device. A series of 3D images, before and after inserting the device shows 27.6% increase to airway volume (From 12.3 to 15.7 cc) and this increase took place especially in the oropharynx region, indicating a reduction of the obstructive potential during the negative inhaling pressure; the tongue volume increased by 17.6% (from 61.8 to 72.7 cc) and the soft palate showed a significant increase. Lowe, in 1990, reported that jawbone repositioning devices can dislodge the jawbone and tongue back and increase the caliper of these upper airways. Lower et al., in 1999, studied an adjustable jawbone positioning device (Klearway) in 38 patients who had SAOS. IAH reduction was from 32.6 to 12.1 events per hour, on average, an IAH reduction to less than 15/hour took place in 80% of patients. An optic fiber videoendoscospy was performed in nine patients with and without the positioning device. No differences were seen in the transversal section of the hypopharinx and oropharynx, But, at the level of the velopharynx, the VAS caliper significantly increased.

Intra-oral devices (AIO) were designed in the 80s and are a clinical type available today for the treatment of sleep respiratory disorders. These devices are used in the oral cavity during sleep with the purpose of preventing the collapse of the oropharynx tissues at the base of the tongue, therefore reducing the obstructive event in the upper airway (VAS). It has been broadly used in patients with snore disorder and apnea syndrome and Obstructive Sleep Apnea Syndrome. This device can be used indefinitely, as it controls the obstructive sleep syndrome rather than being a definite cure. AIO is a type of treatment that has been used because it is simple and not invasive. 1934 is the date the first report of an AIO being used as a type of treatment for jawbone disorder and VAS obstruction. Bruxism is defined as a habitual mouth movement disorder that is characterized by grinding the teeth during sleep, and classified as a sleep movement disorder (AASM/INCREASED—International Classification of Sleep deprivation disorders, 2005). AIO can be used in people with bruxism and can become an effective form of treatment.

### DESCRIPTION OF THE PRIOR ART

Today, a lot has been said about sleep disorders, especially their consequences: low performance at work, difficult learning, transit accidents and workplace incidents, high blood pressure, adversely affected growth rate for children, increased weight, infarction, marital separation, among others. As people become better informed, there is an increased demand for diagnosis and treatment. AIO is a form of treatment, the most comfortable available. In surveys carried out about the state of art, especially among social classes A61C, A62B, A61F, A61M, several patient-related documents have been found for intra-oral devices used to treat snore, apnea and bruxism.

Patent BR 9301908 is related to a device for apnea and snore that is similar to a dental device. This device is manufactured with a smooth acrylic piece in the external side and rugged in the inside, used to minimize snore and apnea disorders. This device has as the disadvantage that its lower part is long and can cause discomfort and an additional disadvantage that it is custom-manufactured, therefore requiring a trained professional to make it.

In PI 0502725-0 (BR), a patent regarding an ANTI-SNORE DEVICE, the chin attachment is designed to keep the device in place during sleep. The device includes straps that work together, with flexibility assured by elastic bands and a fitting system to set the tension and position the device to the patient. This device has the disadvantage of being aesthetically compromising, and the additional disadvantage of only keeping the mouth shut, therefore not eliminating the real causes leading to the obstructions. Compared to BR 0203601, it is better because it is adjustable.

Patent PI 0605955-4 (BR) is for a magnetic device built with a U-shaped module measuring 2 (two) millimeter diameter and 10 (ten) millimeter long, where one side is inserted in the right nostril and the other on the left nostril, and the ends have a receptacle measuring 4 millimeter diameter where high-density magnetic chips are held. The device is manufactured with atoxic PVC plastic (vinylpolychloride). This device has the disadvantage of opening the nose inlet only a little and an additional disadvantage of not eliminating the true obstruction points in the airway and base of the tong.

Patent PI 0606019-6 (BR) is for an electromagnetic device built in atoxic plastic, it has an electromagnetic chip (highly energized magnet) produced with rare elements from nature, such as neodymium (nd) and boron (b) which produces a high magnetic strength on muscular tissues, hardening them and preventing them from relaxing, therefore inhibiting the production of sound vibration and snore. This device has the disadvantage of opening just a little at the nose inlet and an additional disadvantage of not eliminating the obstruction caused when the tongue relaxes, because the tongue is the strongest muscle, therefore adversely affecting the planned result.

Patent PI0603601-5 (BR) reports an auxiliary electronic device to control snore. It is an electronic hand-held device. It has a detection method (microphone sensor) by capturing the sound or vibration from the patient snore and the transmission of signal through the control range and the vibratory or touch device, therefore promoting a small vibration or small touch directly on the patient body, which can interrupt the process where snore occurs. This device has the disadvantage of causing discomfort to the patient with its vibration and an additional disadvantage of awakening the person and interrupt sleep, something that is inconceivable, because its purpose is to improve sleep.

Patent MU8603012-4 (BR) is for an intraoral device for the treatment of primary snore and AOS, including two upper plates and a lower plate made with polystyrene, vacuum-molded in an anatomic shape for the teeth and soft tissues, two helicoid springs manufactured with stainless steel and parts in acrylic resin to join the stainless steel springs and the upper and lower polystyrene plates. This device has the disadvantage of being uncomfortable, in addition to the material with which it is manufactured.

This patent PI0503601-1 (BR) is for an intraoral device for the treatment of snore and apnea disorders which is configured by two upper and lower acrylic plates interconnected by stabilizing arcs having dental screws in their outside. This device has the disadvantage of causing the jawbone to rotate.

Patent MU8500840-0 (BR) teaches a device is enhanced for anti-snore properties, with circumferential retention and opposing clamps around all upper and lower dental parts, manufactured with orthodontic wire. The two parts are articulated by intermaxillae elastic bands ( 3/16, ⅛ or combinations) which are attached to the springs and orthodontic loops that are adapted for the region of the upper canines and lower molars. This device has the disadvantage of not promoting any advancement of the jawbone with the control. Because intermaxillae elastic bands, when in contact with the saliva, lose their properties.

It was long needed to learn the answer regarding the AIO treatment by the patient before manufacturing the final AIO, because it is expensive and is produced in a handcrafted fashion. The device is disposable and allows the patient to perform a polysonogram test for treatment control. This test is performed with the patient wearing the AIO. Additionally, otorhinolaryngological tests can be performed with the device positioned for assessing the point of obstruction of upper airways (VAS) and the base of the tong. Because its use will be longer, as it provides for the possibility of testing, the requestor is concerned with the material; it can be biodegradable or allow sterilization. Advantages include: 1) allowing the patient to perform an adaptation test; 2) protecting the patient from high costs if he/she cannot adapt; 3) it also helps people who travel and can eventually forget to take the device (they can use a product that does not make them feel they are being a nuisance); 4) patients who are under another treatment can also benefit from this device and replace it for a few days, for instance when they are in places where they don't have electric power (some devices used for treatment need power to operate); 5) during airplane or bus trips; 6) because materials used to manufacture the device are disposable or can be sterilized; 7) they can be purchased more quickly; 8) health professionals can assess results from treatment types before establishing which treatment will be established for the patient.

### SUMMARY OF THE INVENTION

In one embodiment the present application provides for a device for treatment of sleep snore and apnea and for use as a mouth protector, comprising a one-piece device having an anatomic shape of dental arcs, teeth and soft tissues of an user, and covering the upper or lower teeth.

In an alternate embodiment the present application provides a disposable device for treatment of sleep snore and apnea, built with an upper part and a lower part for previously designed treatments. The device may be manufactured as a two-part piece or as a single piece; and is fit to position by the patient or trained professional, therefore allowing progressive anterior-posterior fits and providing for side movements. It can be manufactured with materials such as plastic, biopolymers, bioplastic, thermoplastic or thermofixed polymers, acrylic, polycarbonate among others. It can be also developed through a chemical, petrochemical, biological process, whether sources are renewable or not. These plastic materials can be polymer or thermoplastic or thermofixed, they can however be biodegradable or non-biodegradable, or have total or partial biodegradable characteristics. This allow sterilization, both chemical (formaldehyde, glutaraldehyde, ethylene oxide, hydrogen peroxide, peracid acid, hydrogen peroxide plasma among others) or physical (saturated pressure vapor, dry heat, ionizing radiation, non-ionizing radiation, among others). Because it is biodegradable, they provide the advantage of being environmentally friendly and can be disposed without any further environmental restrictions.

The present invention seeks to provide device for treatment of sleep snore and apnea and for use as a mouth protector, the device comprising: (a) an upper part having an anatomic shape of dental arcs, teeth and soft tissues of an user, and covering the upper teeth, and (b) a lower part having an anatomic shape of dental arcs, teeth and soft tissues of the user, and covering the lower teeth; the upper and lower parts are connected, forming a one-piece device, the connection constructed and arranged to provide for sidewise, anterior-posterior and progressive advancements movement of the parts. The parts are connected by an occlusal connection, lateral connection, vestibular connection, frontal connection, internal connection or external connection, enabling progressive anterior-posterior fits and providing for side movements.

### BRIEF DESCRIPTION OF THE DRAWINGS

FIG. 1 is a view of an upper part of a single piece device.

FIG. 2 is a view of a bottom part of a single piece device.

FIG. 3 is a perspective view of a two-part piece joint device.

FIG. 4 is a perspective side view of a two-part piece separated device.);

FIG. 5 is a front view of a two-part separated piece joined device.

FIG. 6 is a front view of a two-part piece joined device, showing some holes.

FIG. 7 is a transversal cut view.

### DETAILED DESCRIPTION OF THE INVENTION

The invention will be described for the purposes of illustration only in connection with certain embodiments; however, it is to be understood that other objects and advantages of the present invention will be made apparent by the following description of the drawings according to the present invention. While a preferred embodiment is disclosed, this is not intended to be limiting. Rather, the general principles set forth herein are considered to be merely illustrative of the scope of the present invention and it is to be further understood that numerous changes may be made without straying from the scope of the present invention.

The present application provides a disposable device for treating sleep snore and sleep apnea. The device can be built with an upper part and a lower part as a two-part piece or as a single piece. The device is easily fit to position by the patient himself/herself or by any trained professional, therefore allowing progressive anterior-posterior fits and providing for side movements and well as anterior-posterior movement.

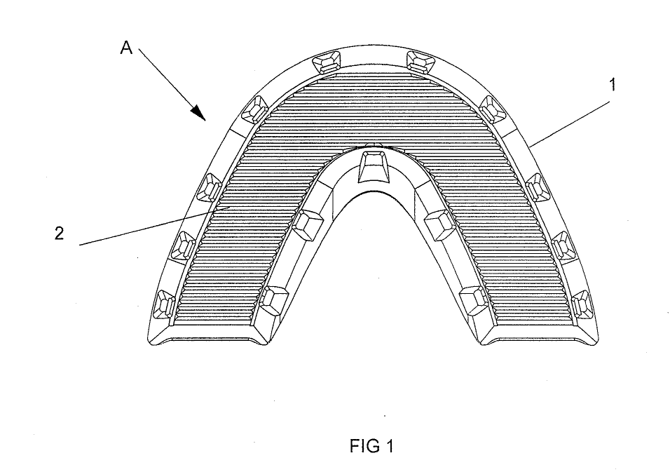

In FIG. 1 an upper part of a single-piece device (A) in the shape of a dental arch (1) is seen, with the site that fits into the teeth in the upper dental arc (2).

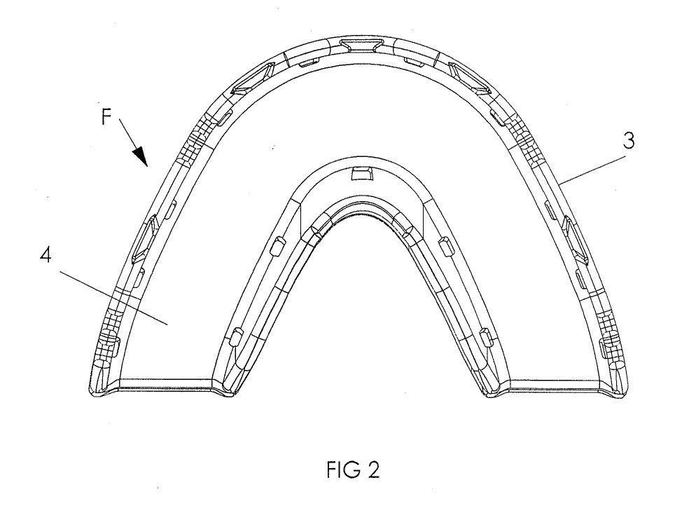

In FIG. 2 a lower part of the single-piece device (F) in the shape of a dental arch (3) is seen, with the site where the teeth will fit into the lower dental arch (4).

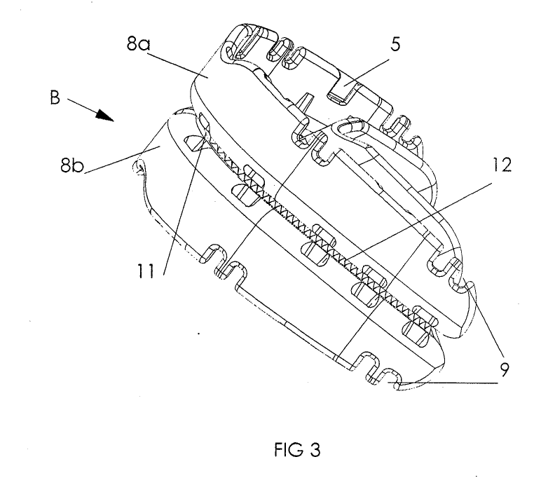

In FIG. 3 a two-part piece joint device (B) has a joint-upper-part (8a) and a joint-lower-part (8b). The device having the anatomic shape of the dental archs, teeth and soft tissues (5). In the occlusal potion (9) includes upper and lower parts covering the upper and lower teeth. The device is constructed and arranged to promote an anterior-posterior movement on the side, and interconnections may be set up on the occlusal, lateral, front, whether the internal or external phase, vestibular, lingual or another position, such as shown in (11) and (12).

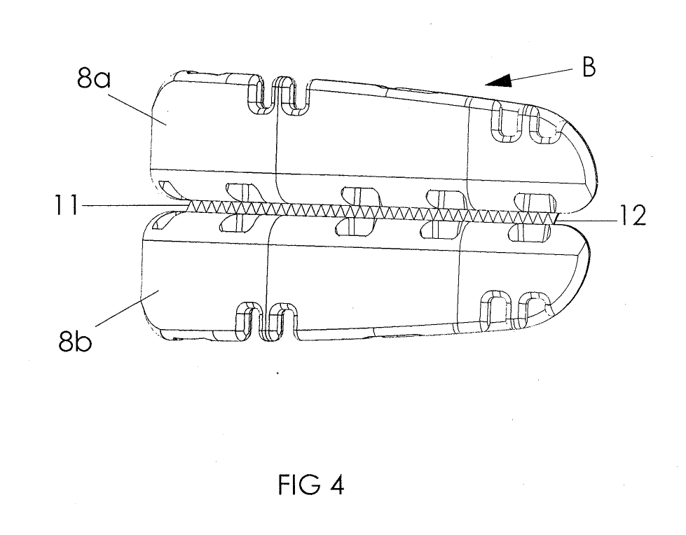

In FIG. 4, it can be seen a side view of the two-part piece separated device (B) where the joint-upper-part (8a) and joint-lower-part (8b) are manufactured separately, linked by linking points, or connections (11) and (12). The linking may be inserted in different positions, such as straight, angled, diagonal, forming geometric figures. There may be one or more support spots in each of the pieces, for fixing the junction.

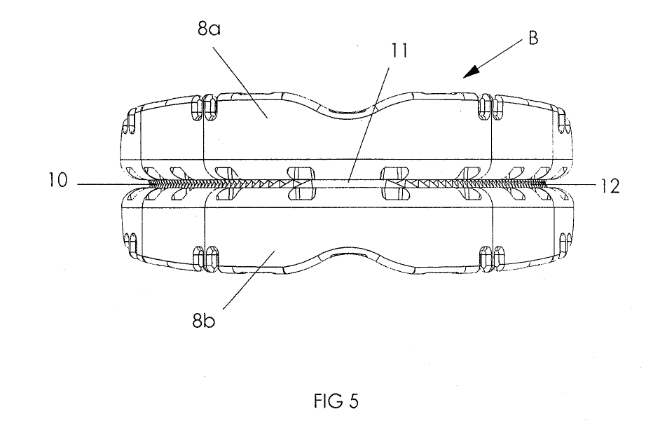

In FIG. 5, it can be seen a front view of the two-part piece separated device (B) where the joint-upper-part (8a) and lower part (8b) are manufactured separately, linked by or connections (10), (11) and (12).

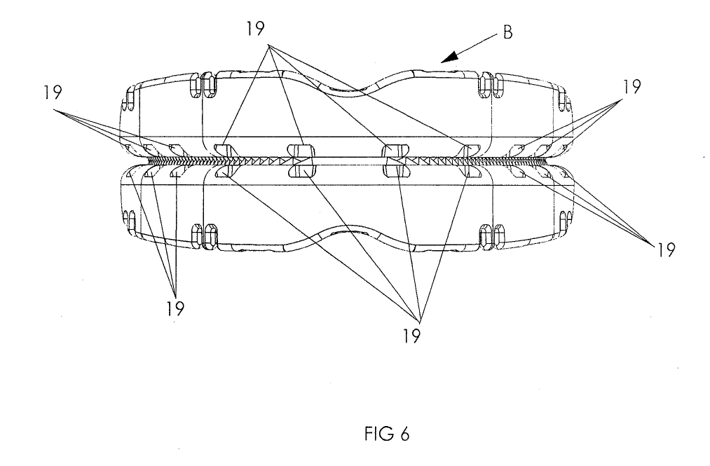

FIG. 6 show a front view of the two-part piece joined device (B), showing holes (19) to provide for airflow and for linkage between the materials.

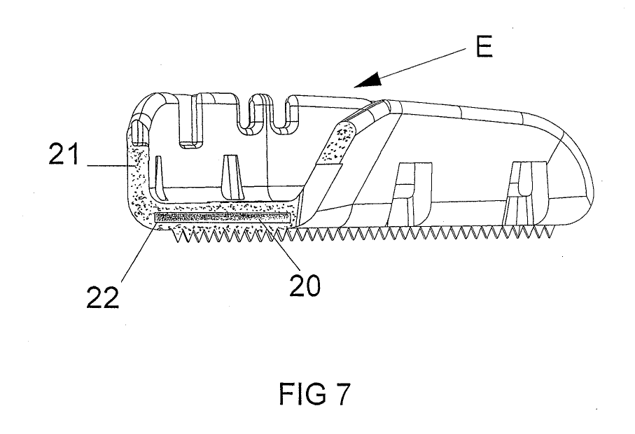

FIG. 7 is a transversal cut view of a single-piece device (E), the interior part of the cavity is more rigid (20) and the exterior part is softer (21) and the connection between the exterior and interior parts (22).

It should be understood that the preferred embodiments mentioned here are merely illustrative of the present invention. Numerous variations in design and use of the present invention may be contemplated in view of the following claims without straying from the intended scope and field of the invention herein disclosed.

1-23. (canceled)

24. A device for treatment of sleep snore and apnea and for use as a mouth protector, the device comprising a one-piece device having an anatomic shape of dental arcs, teeth and soft tissues of an user, and covering the upper or lower teeth.

25. A device for treatment of sleep snore and apnea and for use as a mouth protector, the device comprising:
(a) an upper part having an anatomic shape of dental arcs, teeth and soft tissues of an user, and covering the upper teeth, and
(b) a lower part having an anatomic shape of dental arcs, teeth and soft tissues of the user, and covering the lower teeth
wherein the upper and lower parts are connected, forming a one-piece device, the connection constructed and arranged to provide for sidewise, anterior-posterior and progressive advancements movement of the parts.

26. The device according to claim 25, wherein the parts are connected by a connection selected from the group consisting of: occlusal connection, lateral connection, vestibular connection, frontal connection, internal connection, external connection and combinations thereof.

27. The device according to claim 25, further comprising multiple holes to provide for airflow.

28. The device according to claim 25, wherein the device is designed any desired size.

29. The device according to claim 25, wherein the parts are manufactured with one layer of the same material, or multiple layers of materials.

30. The device according to claim 25, wherein the parts are manufactured in a thermoplastic plastic material, including polymer or biopolymer.

31. The device according to claim 25, wherein the parts are manufactured in a plastic material through a chemical, biological or petrochemical production process.

32. The device according to claim 25, wherein the parts are manufactured in a plastic material originated from vegetable or fossil raw materials.

33. The device according to claim 25, wherein the parts are manufactured in biodegradable or non-biodegradable material.

34. The device according to claim 25, wherein the parts provide for both chemical sterilization and physical sterilization.

35. The device according to claim 34, wherein the chemical sterilization is selected from the group consisting of: formaldehyde, glutaraldehyde, ethylene oxide, hydrogen peroxide, peracid acid and hydrogen peroxide plasma.

36. The device according to claim 34, wherein the physical sterilization is selected from the group consisting of: saturated pressure vapor, dry heat, ionizing radiation and non-ionizing radiation.

37. The device according to claim 25, wherein the parts are manufactured in a handcrafted fashion.

38. The device according to claim 25, further comprising multiple holes to provide for linkage between the materials.

39. The device according to claim 24, wherein the device is designed any desired size.

40. The device according to claim 24, wherein the parts are manufactured with one layer of the same material, or multiple layers of materials.
